(19) United States
(12) Patent Application Publication
(10) Pub. No.: US 2024/0159729 A1
(43) Pub. Date: May 16, 2024

(54) A METHOD OF MEASURING SOIL

(71) Applicant: AUSTRALIAN NATURAL CAPITAL (IP) PTY LTD, Brisbane, Queensland (AU)

(72) Inventor: Benjamin Joe LODGE, Brisbane, Queensland (AU)

(21) Appl. No.: 18/550,863

(22) PCT Filed: Mar. 15, 2022
(86) PCT No.: PCT/AU2022/050220

§ 371 Date: Sep. 15, 2023

(30) Foreign Application Priority Data
Mar. 15, 2021 (AU) 2021900744
Aug. 25, 2021 (AU) 2021221611

(51) Int. Cl. G01N 33/24

(52) U.S. Cl. CPC G01N 33/24

(57) ABSTRACT

A method of measuring soil, the method including the steps of assessing the characteristics of a landscape under inspection to identify one or more feature groups of the landscape under inspection and the amount of area of land of each of the feature groups; identifying sampling locations in each of the feature groups; undertaking soil sampling at each identified sampling locations to calculate the content of the feature of the soil in each of the feature groups; and calculating the content of the feature of the landscape under inspection using the calculated content of a feature of the soil of each of the feature groups and the amount of area of land of each feature group as a proportion of the total area of the landscape under inspection.

### FIELD OF THE INVENTION

The present disclosure relates to a method of assessing soil. In particular, although not exclusively, the invention relates to a method of measuring the amount of carbon within soil.

### BACKGROUND OF THE INVENTION

Carbon sequestration is the long-term storage of carbon in plants, soils, geologic formations, and the ocean, and commonly the long-term removal, capture or sequestration of carbon dioxide from the atmosphere to slow or reverse atmospheric CO2 pollution and to mitigate or reverse global warming. Carbon dioxide is naturally captured from the atmosphere through biological, chemical, and physical processes. That process also regenerates the soil and allows for increased land productivity.

Due to the introduction of various government and market driven financial schemes and incentives, such as carbon credits and the like, it is desirable to measure the change in carbon stocks in soil over time for landholders and other entities participating in these schemes.

Soil carbon concentrations can be highly variable on regional, local and micro scales and this causes difficulty when trying to determine representative stocks of soil carbon, which is a necessary step to certifying how much carbon has been sequestered over time.

Many known methods are crude and apply a grid based or random sampling techniques to provide an indication of the concentration of carbon in the soil. Such techniques do not adequately account for the variability of carbon at a regional, local or micro scale within an area of land.

Alternatively, attempts have been made to accurately calculate carbon soil stocks in a landscape under inspection and accommodate for the variability by increasing the amount of sampling and analysis undertaken until the dataset obtained is large enough to statistically accommodate for the carbon concentration variability. This dramatically increases the costs of the physical sampling program.

This difficulty with assessing change in carbon stocks in soil over time has decreased the accessibility of carbon sequestration schemes due to the inaccurate nature of the sampling technique and/or the high cost of having the sampling carried out to get an accurate result sufficient for carbon credit schemes and/or other financially motivated schemes.

It is an object to overcome or at least ameliorate one or more of the deficiencies of the prior art described above and/or provide the consumer with a useful commercial choice.

### SUMMARY OF THE INVENTION

In one form, although it need not be the only form or indeed the broadest form, the invention relates to method of measuring soil, the method including the steps of:
assessing the characteristics of a landscape under inspection to identify one or more feature groups of the landscape under inspection and the amount of area of land of each of the feature groups;
identifying sampling requirements in each of the feature groups;
undertaking soil sampling for each identified sampling requirements to calculate the content of the feature of the soil in each of the feature groups; and
calculating the content of the feature of the landscape under inspection using the calculated content of a feature of the soil of each of the feature groups.

Preferably, the feature is carbon.

Suitably, the step of calculating the content of the feature of the landscape under inspection further includes use of the amount of area of land of each feature group as a proportion of the total area of the landscape under inspection.

Alternatively, the step of calculating the content of the feature of the landscape under inspection further includes use of the amount of area of land of each feature group as a portion of the total area of the landscape under inspection.

Optionally, identification of one or more feature groups of the landscape under inspection includes incorporating one or more feature groups that consist of different observable features that are incorporated based on analysis of statistically similar characteristics of the content of the feature of the soil.

Suitably, the step of undertaking soil sampling at each identified sampling locations to calculate the content of the feature of the soil in each feature group is undertaken by soil sampling in the field.

Optionally, the step of undertaking soil sampling at each identified sampling locations to calculate the content of the feature of the soil in each feature group is undertaken by interrogation of a data store having the content of the feature of the soil stored therein collected from previous field sampling.

Optionally, the step of undertaking soil sampling at each identified sampling locations to calculate the content of the feature of the soil in each feature group is undertaken by soil sampling in the field in conjunction with interrogation of a data store having the content of the feature of the soil stored therein collected from previous field sampling.

Alternatively, the feature is a mineral, chemical, biological or physical property.

Suitably, the step of identifying one or more feature groups of the landscape under inspection and the amount of area of land of each of the feature group includes calculating the amount of land of each feature group as a proportion or portion of the total area of land of the landscape under inspection. Optionally, this also includes calculating the vertical proportion or portion of the below ground strata of the soil of each feature group to calculate a feature proportion or portion of the landscape under inspection. In a preferred form, that feature proportion or portion may be represented as a volume.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1 shows a flowchart of a method of measuring soil according to an embodiment of the invention;

FIG. 2 shows a schematic of a landscape under inspection to which the method is applied; and

FIG. 3 shows the table representing data that is produced as a result of flowchart shown in FIG. 1.

### DETAILED DESCRIPTION OF THE INVENTION

The present invention pertains to a method of measuring soil. An embodiment of the present invention is described below with reference to sampling soil to determine concentrations of carbon within that soil. A skilled person will understand that method may equally be applicable to concentrations of other chemical elements in the soil and/or other characteristics of the soil such as degrees of contamination or the like.

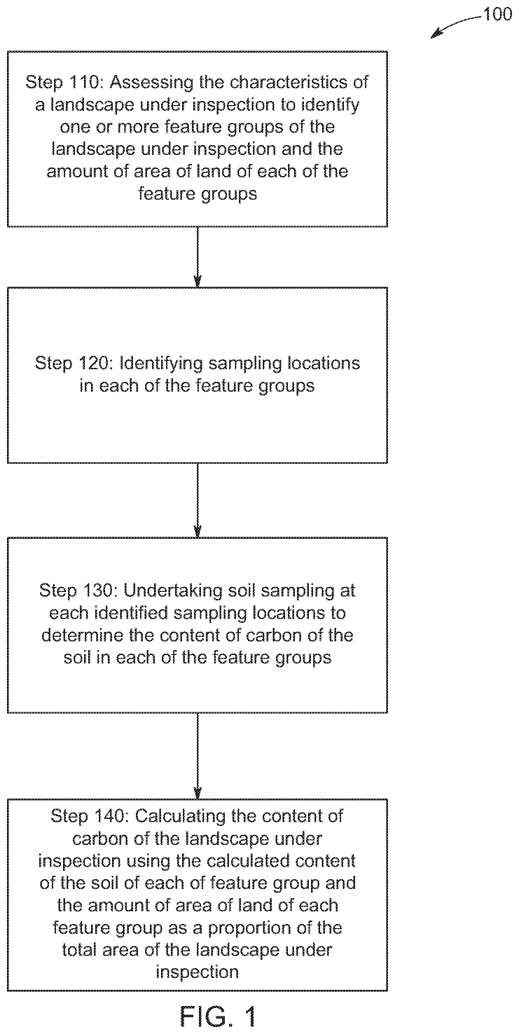

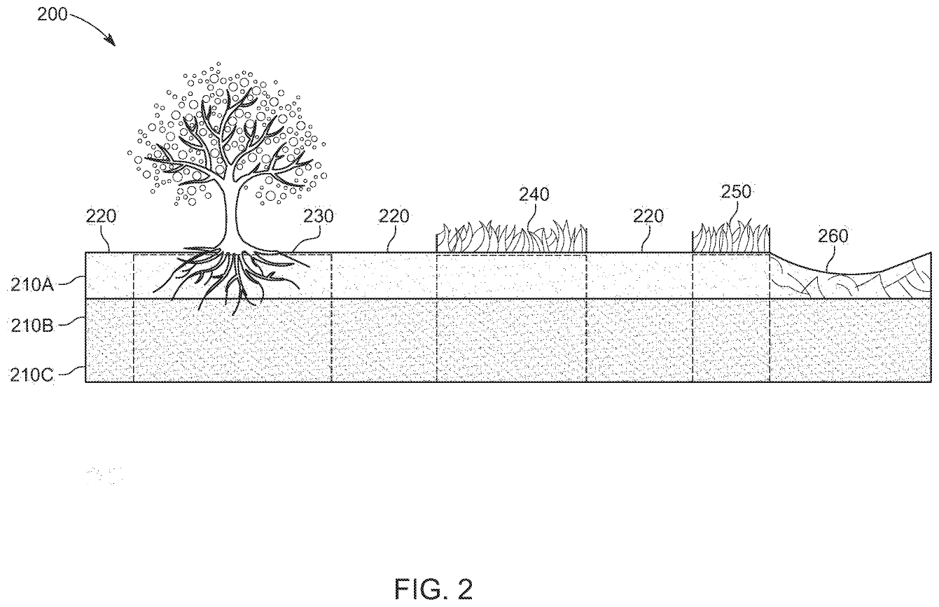

FIG. 1 shows a flowchart of a method 100 of sampling soil according to an embodiment of the invention. FIG. 2 shows a schematic of a landscape 200 under inspection to which the method is applied.

Method 100 has the step 110 of assessing the characteristics of the landscape 200 under inspection to identify one or more feature groups of the landscape 200 and the amount of area of land each of the feature groups takes up within the landscape 200. Areas of land in the landscape 200 are grouped together based on observable features within the landscape 200 that have the capacity to influence the variability of the concentrations of carbon and/or the ability to sequester carbon. Factors that are considered in this step include the nature, extent and variability of soil type, grasses, ground vegetation, ground cover, bare ground, climate, chemistry, biology, surface presentation of the land, topography including localised depressions, soil density, gravel content, water movement, water courses, water bodies, vegetation species, canopy cover, rock outcrops, infrastructure land management practices, natural occurrences and other such factors as will be appreciated by a person skilled in the art.

This step may also incorporate together in a single feature group one or more of these observable features based on an analysis of statistically similar characteristics of the soil content, which in the embodiment is concentrations of carbon. For example, areas of land that have canopy cover may have similar carbon concentrations and sequestration ability, as rock outcrops based on a statistical analysis of ground with those types of observable characteristics. As such, in this example, those two areas of land would be grouped together as a single feature group.

With reference to the embodiment in FIG. 2, identification of features in the landscape 200 has occurred to identify features groups as follows: bare ground 220, common tree type coverage 230, grass coverage of a first pasture type 240, grass coverage of a second pasture type 250 and surface depression 260.

A skilled addressee will recognise other features may be identified such as shrub coverage, rocky outcrops, water bodies, man made coverage such as roads and the like.

Furthermore, different below ground strata 210A, 210B and 210C are also identified as feature groups. A skilled addressee will appreciate that there may be different number of below ground strata levels for each surface feature. Whilst three are shown in the embodiment there may be variable numbers based on location on the landscape 200.

The feature groups in the embodiment have been identified with reference to identifying a feature of the soil in the landscape that is carbon content. As such, the feature groups identified above have been identified based upon their common characteristics associated with carbon content and/or their ability to sequester carbon. A skilled person will appreciate that when the invention is applied to measure, including for other characteristics of a landscape, feature groups will be assessed based upon the relevant factors in that circumstance.

Once the feature groups 220-260 have been identified the area of land assigned of each feature group is then calculated. In the embodiment, the area of land of each feature group 220-260 is calculated and represented as a proportion of the total area of land of the landscape 200.

The step of assessing the characteristics of the landscape 200 to identify the feature groups 220-260 and the area of land of each feature group 220-260 is carried out using suitable means of landscape assessment including review of available literature and data groups associated with the landscape 200, geospatial analysis and modelling, field analysis and observation, photography, proximate sensing, sampling and analysis (such as drilling and the like), remote sensing, ground truthing, quality assurance, database management and through the use of georeferenced drones, statistical analysis, satellites and other visual inspection mechanisms and/or other like landscape surveying techniques.

Suitably, this step also involves calculating the vertical proportion of the below ground strata 210A-C of the soil of each feature group 220-260 of the landscape 200 to calculate a feature proportion of each feature group 220-260 as discussed further below.

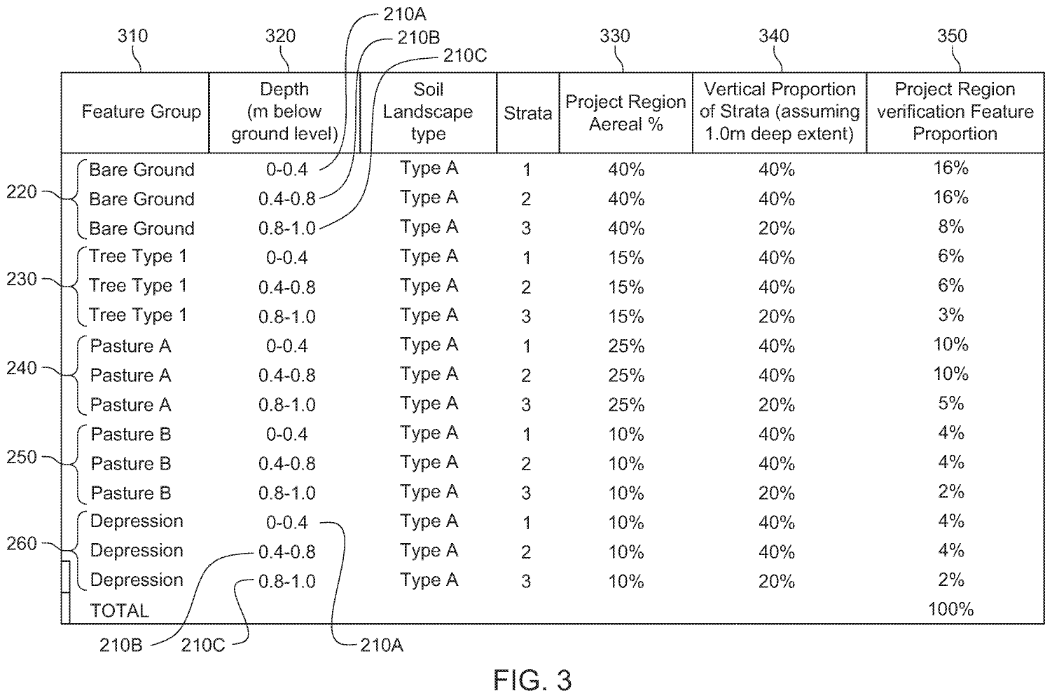

FIG. 3 shows a table 300 which represents the data as an outcome of the step 110 discussed above. In particular, table 300 has a row showing each of the feature groups 220 to 260, as shown in column 310, broken out to accommodate for the vertical depth of each of the below ground strata 210A-C as shown in column 320. The area of that feature group 220 to 260 as a portion for the total area of the landscape 200 is shown in column 330.

The vertical proportion of the below ground strata 210A-210C of each feature group 220-260 is shown in column 340. The feature portion, shown in column 350, is calculated by taking the vertical proportion 210A, 210B and 210C respectively of the relevant feature group 22-260 as a fraction of the area of each feature group 220-260 as a proportion of the total area of the landscape 200 under consideration.

In embodiments with different number of strata for each feature groups, or non uniform strata vertical proportion or portion, the feature proportion or portion is suitably expressed as a volume.

The feature portion is discussed in greater detail below.

Referring again to FIG. 1, in step 120 the sample locations within each feature group 220-260 are then identified. Various considerations are made as to sample locations within each feature group 220-260 such as physical access availability, statistical significance, randomness, compositing, modelling of land to determine sample locations (and depth).

In step 130, soil sampling, sample preparation, and analysis is undertaken to determine the content of carbon in each of the feature groups 220-260. That process includes field sampling (for example drilling) analysis and calculation to thereby enable calculation of the amount of carbon content in each of the soil samples from the sample locations within the relevant feature group 220-260 and inferring carbon content in the feature group 220-260.

In an alternative embodiment, the step 130 of soil sampling, sample preparation and analysis to determine the content of carbon in each of the feature groups 220-260 is undertaken by interrogation of a data store having stored therein content of carbon from previous field sampling. In this alternative embodiment we are identifying the sampling requirements in each of the feature groups and then undertaking consequential calculation of the content of the feature of the soil in each of the feature groups by way of interrogation of the data store.

In still a further embodiment that process involves statistical analysis of like feature groups rather than actual sampling from the feature group 220-260.

In still a further form, physical sampling is undertaken as described in combination with interrogation of a data store as previously described.

In step 140, the content of the carbon of the landscape 200 under inspection is then calculated by using the feature proportion for the relevant feature group as weighting factor against the carbon content measured in the feature group from step 130 and summing those values.

In an alternative embodiment, the content of the carbon of the landscape 200 under inspection is calculated using other means such as summing the carbon content from each of the feature groups and other like means.

By identifying feature groups and undertaking measurement in this way to allow selection of physical sampling locations and then, in some embodiments, physically sampling (such as by drilling at those selected locations) the cost of soil sampling and analysis is reduced as high density exhaustive sampling programs are obviated whilst still maintaining a high degree of confidence in the data produced.

In the embodiment, the method 100 is repeated at various points in time so as to determine the change in total carbon content or stock in the landscape under inspection to determine the value of carbon that has been sequestered in the soil over time.

In some embodiments of the invention, modelling within a feature group may be used so as the carbon content or stock of the soil can be calculated at various points within the feature group as the area of land approaches the border with a different feature group. In this way, significant step changes in carbon stock calculations can be avoided at the boundary between feature groups. Rather, the carbon stock and sequestration ability of the soil within a particular feature group can be tapered from the physical sampling location in the feature group to the boundary of the feature group.

Whilst the embodiment has been described with reference to the feature being carbon content in the soil, the method may also be applied to measure other features that have concentrations that are highly variable on regional, local and micro scales. Other examples include soil concentration from fuel terminals and lead distribution in shotgun ranges.

By “about” or “approximate” and their grammatically equivalent expressions is meant a quantity, level, value, number, frequency, percentage, dimension, size, amount, abundance, concentration, weight or length that varies by as much 15, 14, 13, 12, 11, 10, 9, 8, 7, 6, 5, 4, 3, 2 or 1% to a reference quantity, level, value, number, frequency, percentage, dimension, size, amount, abundance, concentration, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present.

1. A method of measuring soil, the method including the steps of:
assessing the characteristics of a landscape under inspection to identify one or more feature groups of the landscape under inspection and the amount of area of land of each of the feature groups;
identifying sampling requirements in each of the feature groups;
undertaking soil sampling for each identified sampling requirement to calculate the content of the feature of the soil in each of the feature groups; and
calculating the content of the feature of the landscape under inspection using the calculated content of a feature of the soil of each of the feature groups.

2. The method of claim 1, wherein the feature is carbon.

3. The method of claim 1, wherein the step of undertaking soil sampling at each identified sampling locations to calculate the content of the feature of the soil in each feature group is undertaken by soil sampling in the field.

4. The method of claim 1, the step of undertaking soil sampling at each identified sampling locations to calculate the content of the feature of the soil in each feature group is undertaken by interrogation of a data store having the content of the feature of the soil stored therein collected from previous field sampling.

5. The method of claim 1, the feature is a mineral, chemical, biological or physical property.

6. The method of claim 1, wherein the step of calculating the content of the feature of the landscape under inspection further includes use of the amount of area of land of each feature group as a proportion or portion of the total area of the landscape under inspection.

7. The method of claim 6, the step of identifying one or more feature groups of the landscape under inspection and the amount of area of land of each of the feature group includes calculating the amount of land of each feature group as a proportion or portion of the total area of land of the landscape under inspection.

8. The method of claim 6, wherein the step of identifying one or more feature groups of the landscape under inspection and the amount of area of land of each of the feature group includes calculating the amount of land of each feature group as a proportion or portion of the total area of land of the landscape under inspection.

9. The method of claim 8, further including the step of calculating the vertical proportion or portion of the below ground strata of the soil of each feature group to calculate a feature proportion or portion of the landscape under inspection.

10. The method of claim 1, wherein the step of undertaking soil sampling involves physical sampling of the soil at locations in the landscape under review as determined by the sampling requirements identified in each of the feature groups.
